(19) United States
(12) Patent Application Publication
(10) Pub. No.: US 2025/0161552 A1
(43) Pub. Date: May 22, 2025

(54) SYSTEMS, DEVICES, AND METHODS FOR NITRIC OXIDE AND NEGATIVE PRESSURE WOUND THERAPY

(71) Applicant: T.J.Smith and Nephew,Limited, Hull (GB)

(72) Inventors: Varuni Rachindra Brownhill, Swanland (GB); Benjamin James Gardner, Hull (GB); Iain Webster, Hull (GB)

(21) Appl. No.: 18/841,636

(22) PCT Filed: Feb. 23, 2023
(86) PCT No.: PCT/EP2023/054504

§ 371 Date: Aug. 26, 2024

(30) Foreign Application Priority Data
Feb. 28, 2022 (GB) 2202725.4

(51) Int. Cl. A61M 1/00

(52) U.S. Cl. CPC A61M 1/94; A61M 1/985; A61M 2202/0275

(57) ABSTRACT

Disclosed embodiments relate to a wound therapy system that can deliver nitric oxide and apply negative pressure to a wound. The wound therapy system can include a wound dressing for placement over the wound, a source of negative pressure connected to the wound dressing, a source of nitric oxide connected to the wound dressing, and a controller configured to regulate nitric oxide delivery and negative pressure application to the wound dressing. The controller can be programmed to deliver various concentrations of nitric oxide to the wound dressing at various times over the course of treatment of the wound. The controller can also be programmed to apply various levels of negative pressure to the wound dressing at various times over the course of treatment of the wound.

### CROSS-REFERENCE TO RELATED APPLICATIONS

This application claims priority to U.K. Provisional Application No. 2202725.4 filed on Feb. 28, 2022; the disclosure of which is hereby incorporated by reference in its entirety.

### BACKGROUND

### Field

Embodiments of the present disclosure relate to systems, devices, and methods for treating a wound, for example with nitric oxide in combination with negative pressure wound therapy.

### Description of the Related Art

Many different types of wound dressings are known for aiding in the healing process of a human or animal. These different types of wound dressings include many different types of materials and layers, for example, gauze, pads, foam pads or multi-layer wound dressings. Negative pressure wound therapy (NPWT), sometimes referred to as vacuum assisted closure, topical negative pressure therapy, or reduced pressure wound therapy, is widely recognized as a beneficial mechanism for improving the healing rate of a wound. Such therapy is applicable to a broad range of wounds such as incisional wounds, open wounds, abdominal wounds, infected wounds, complex wounds, and the like. The formation of infection-free healing tissue in a short period of time is essential to minimize complications. While NPWT can be used successfully to drain excess exudate, reduce tissue oedema, stimulate the formation of granulation tissue and stimulate angiogenesis, managing the wound microbiology can be challenging. Increasingly, the use of NPWT with antimicrobial products such as silver dressings have been put forward but these do not always provide an effective management of infected wounds. In addition, device failures and frequent dressing changes can lead to increasing the risk of developing wound infection. Biofilms are also a known problem in soft tissue which can lead to increased risk of biofilm-related infections and also delayed healing.

Instillation therapy, which involves the use of antiseptics or antibiotics that are irrigated into the wound, is sometimes used with NPWT to manage infected wounds. During instillation therapy, a desired solution is pumped into foam placed within the wound via a dedicated tubing system and then, after a set time during which the instillation is left to take effect with no suction applied, the solution is removed by suction after which NPWT can begin. Several challenges exist, however, with this combined approach. Dwelling times of the instillation therapy solution can be too short, making this approach ineffective at killing pathogens. Conversely, dwelling times can be too long, which can be cytotoxic to healing and healthy tissue. Furthermore, biofilms deep within soft tissue may not be fully targeted by the solutions used. Thus, improved ways of managing the wound microbiology during NPWT are needed.

A separate approach to positively influencing wound healing is by the delivery of nitric oxide (NO) to a wound. Nitric oxide is a well-known molecule with multiple biological functions. For example, nitric oxide influences blood vessel vasodilation, stimulates angiogenesis, influences the host immune response, and demonstrates potent, broad spectrum antimicrobial activity and anti-biofilm activity. Due to these multiple roles, NO demonstrates a potent effect on tissue and increased amounts of NO may support the acceleration of healing in wounds, particularly chronic wounds.

Additionally, diabetic patients often have lower levels of nitric oxide as compared to healthy patients, and diminished supply of nitric oxide in diabetic patients is a compounding factor in a healing chronic ulcer. Diminished supply of nitric oxide may lead to vascular damage, such as endothelial dysfunction and vascular inflammation. Vascular damage may also lead to decreased blood flow to the extremities, thereby potentially causing the diabetic patient to be more likely to develop neuropathy and non-healing ulcers, and to be at a greater risk for lower limb amputation.

Under normal conditions, nitric oxide, a free radical, is short-lived and converted to a more stable chemical species within seconds of production. Thus, for example, if gaseous nitric oxide contacts air, the gaseous nitric oxide will be rapidly oxidized to generate nitrogen dioxide (NO₂). Accordingly, it may be difficult to maintain high concentrations of nitric oxide within a wound dressing or other similar structure for a prolonged period of time. Consequently, there is a need for improved mechanisms of delivering an effective dose of nitric oxide to a wound. Of particular interest are mechanisms of delivering nitric oxide in combination with use of a wound dressing, particularly a negative pressure wound dressing and/or while undergoing NPWT and/or other appropriate therapies.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods.

Disclosed herein is a negative pressure wound therapy system configured for delivery of nitric oxide, including a wound dressing configured for placement over a wound, a source of negative pressure configured to be connected to the wound dressing, a source of nitric oxide configured to be connected to the wound dressing, and a controller configured to regulate nitric oxide delivery and negative pressure application to the wound dressing. The controller is programmed to: apply negative pressure to the wound dressing to cause a pressure level within the wound dressing to reach a first negative pressure level, pause or reduce application of negative pressure to the wound dressing and deliver nitric oxide to the wound dressing to increase the pressure level within the wound dressing from the first negative pressure level to a second negative pressure level, pause or reduce delivery of nitric oxide and apply negative pressure to the wound dressing until a third negative pressure level within the wound dressing between the first negative pressure level and the second negative pressure level is reached, pause or reduce application of negative pressure to the wound, and apply negative pressure to the wound dressing to evacuate nitric oxide from the wound dressing and to cause the pressure level within the wound dressing to decrease toward the first negative pressure level.

In the above system or in other embodiments as described herein, one or more of the following features may also be provided. In some embodiments, when the third negative pressure level within the wound dressing is reached, application of negative pressure to the wound dressing comprises maintaining said third negative pressure level within the wound dressing until negative pressure is applied to the wound dressing to evacuate nitric oxide from the wound dressing. In some embodiments, when the third negative pressure level within the wound dressing is reached, the controller is further programmed to deliver nitric oxide to the wound dressing to maintain a concentration of nitric oxide within the wound dressing. In some embodiments, the source of nitric oxide comprises a source of pressurized nitric oxide gas. In some embodiments, the source of pressurized nitric oxide gas comprises one or more cartridges configured to store and release said pressurized nitric oxide gas. In some embodiments, the source of nitric oxide comprises a nitric oxide generator. In some embodiments, the nitric oxide generator comprises a plasma generator. In some embodiments, the plasma generator produces nitric oxide from air or an oxygen rich gas mixture. In some embodiments, the system further comprises a valve configured to control the delivery of nitric oxide to the dressing. In some embodiments, the controller is in electrical communication with said valve, and the controller is configured to operate the valve to regulate nitric oxide delivery to the wound dressing. In some embodiments, the valve comprises a solenoid valve. In some embodiments, the system further comprises an exhaust valve configured to control a supply of atmospheric pressure to the wound dressing. In some embodiments, the controller is in electrical communication with said exhaust valve, and the controller is configured to operate the exhaust valve to regulate the supply of atmospheric pressure to the wound dressing. In some embodiments, applying negative pressure to the wound dressing includes opening said exhaust valve. In some embodiments, the exhaust valve comprises a solenoid valve. In some embodiments, the system further comprises a housing configured to house the source of negative pressure, the source of nitric oxide, and the controller. In some embodiments, said housing is configured to be wearable and/or portable. In some embodiments, the system further comprises a connector portion configured to fluidly connect the source of negative pressure and the source of nitric oxide to the wound dressing. In some embodiments, said wound dressing comprises the source of negative pressure, the source of nitric oxide, and the controller. In some embodiments, the system further comprises a pressure sensor configured to measure the pressure level within the wound dressing. In some embodiments, the controller is in electrical communication with said pressure sensor, and the controller is further programmed to determine the pressure level within the wound dressing based on the pressure level measured by said pressure sensor. In some embodiments, the controller is further programmed to determine a concentration of nitric oxide within the wound dressing based on said determined pressure level. In some embodiments, the controller is further programmed to determine an amount of time nitric oxide is delivered to the wound dressing and determine a concentration of nitric oxide within the dressing based on said amount of time. In some embodiments, the system further comprises a nitric oxide sensor configured to measure a concentration of nitric oxide within the wound dressing. In some embodiments, the nitric oxide sensor is not located within the wound dressing. In some embodiments, the controller is in electrical communication with said nitric oxide sensor, and the controller is further programmed to determine the concentration of nitric oxide within the wound dressing based on the concentration of nitric oxide measured by said nitric oxide sensor. In some embodiments, the system further comprises one or more visual indicators configured to generate illumination visible to a user of the system and in electrical communication with the controller, and the controller is further programmed to compare said determined concentration of nitric oxide within the wound dressing to one or more thresholds and instruct the one or more visual indicators to turn on or off based on said comparison. In some embodiments, the system further comprises an electronic display in electrical communication with the controller, and the controller is further programmed to provide on said electronic display a graphical representation of the determined concentration of nitric oxide within the wound dressing. In some embodiments, the controller is further programmed to compare said determined concentration of nitric oxide within the wound dressing to one or more thresholds and provide on said electronic display an alarm based on said comparison. In some embodiments, the system further comprises a speaker configured to generate sound audible to a user of the system and in electrical communication with the controller, and the controller is further programmed to compare said determined concentration of nitric oxide within the wound dressing to one or more thresholds and instruct the speaker to produce sound based on said comparison. In some embodiments, the controller is further programmed to compare said determined concentration of nitric oxide within the wound dressing to one or more thresholds and alarm a user of the system based on said comparison. In some embodiments, the controller is further programmed to determine a volume of the system. In some embodiments, the volume of the system includes a volume of the wound enclosed by the wound dressing. In some embodiments, the volume of the system is determined based on a rate of pressure change or a pressure change in the system over time. In some embodiments, the volume of the system is determined when negative pressure is applied to the wound dressing. In some embodiments, the system further comprises one or more indicators configured to indicate a status of the system. In some embodiments, the first negative pressure level comprises a range including or between −80 mmhg and −200 mmhg. In some embodiments, the second negative pressure level comprises a range including or between −20 mmhg and −120 mmhg. In some embodiments, the third negative pressure level comprises a range including or between −40 mmhg and −200 mmhg. In some embodiments, a concentration of nitric oxide within the wound dressing at the first pressure level comprises a range including or between 0 ppm and 30 ppm. In some embodiments, a concentration of nitric oxide within the wound dressing at the first pressure level is 30 ppm or less. In some embodiments, a concentration of nitric oxide within the wound dressing at the second pressure level comprises a range including or between 80 ppm and 500 ppm. In some embodiments, a concentration of nitric oxide within the wound dressing at the second pressure level is 80 ppm or greater. In some embodiments, a concentration of nitric oxide within the wound dressing at the third pressure level comprises a range including or between 5 ppm and 80 ppm. In some embodiments, a concentration of nitric oxide within the wound dressing at the third pressure level is between a factor of 5 and a factor of 15 less than a concentration of nitric oxide within the wound dressing at the second pressure level. In some embodiments, a concentration of nitric oxide within the wound dressing at the third pressure level is a factor of 10 less than a concentration of nitric oxide within the wound dressing at the second pressure level. In some embodiments, the second negative pressure level is maintained for one hour or less. In some embodiments, the second negative pressure level is maintained for 20 minutes or less. In some embodiments, the second negative pressure level is maintained for 5 minutes or less.

Disclosed herein is a negative pressure wound therapy system configured for delivery of nitric oxide, comprising a wound dressing configured for placement over a wound, a source of negative pressure configured to be connected to the wound dressing, a source of nitric oxide configured to be connected to the wound dressing, and a controller configured to regulate nitric oxide delivery and negative pressure application to the wound dressing. The controller is programmed to: apply negative pressure to the wound dressing at a first time to cause a pressure level within the wound dressing to reach a first negative pressure level, pause or reduce application of negative pressure to the wound dressing and deliver nitric oxide to the wound dressing at a second time until a first nitric oxide concentration is reached at a third time and to cause the pressure level within the wound dressing to increase from the first negative pressure level to a second negative pressure level, pause or reduce delivery of nitric oxide at the third time, apply negative pressure to the wound dressing at a fourth time until a second nitric oxide concentration is reached at a fifth time and a third negative pressure level within the wound dressing between the first negative pressure level and the second negative pressure level is reached, pause or reduce application of negative pressure to the wound at the fifth time, and apply negative pressure to the wound dressing at a sixth time to evacuate nitric oxide from the wound dressing and to cause the pressure level within the wound dressing to decrease toward the first negative pressure level.

In the above system or in other embodiments as described herein, one or more of the following features may also be provided. In some embodiments, the application of negative pressure to the wound is paused or reduced at the fifth time to maintain said second nitric oxide concentration within the wound dressing. In some embodiments, the controller is further programmed to deliver nitric oxide to the wound dressing between the third and fifth times to maintain the first nitric oxide concentration within the wound dressing. In some embodiments, the controller is further programmed to deliver nitric oxide to the wound dressing between the fifth and sixth times to maintain the second nitric oxide concentration within the wound dressing. In some embodiments, the first negative pressure level comprises a range including or between −80 mmhg and −200 mmhg. In some embodiments, the second negative pressure level comprises a range including or between −20 mmhg and −120 mmhg. In some embodiments, the third negative pressure level comprises a range including or between −40 mmhg and −200 mmhg. In some embodiments, the first nitric oxide concentration comprises a range including or between 80 ppm and 500 ppm. In some embodiments, the first nitric oxide concentration is 80 ppm or greater. In some embodiments, the second nitric oxide concentration comprises a range including or between 5 ppm and 80 ppm. In some embodiments, the second nitric oxide concentration is 80 ppm or less. In some embodiments, the second nitric oxide concentration is between a factor of 5 and a factor of 15 less than the first nitric oxide concentration. In some embodiments, the second nitric oxide concentration is a factor of 10 less than the first nitric oxide concentration. In some embodiments, the second negative pressure level is maintained for one hour or less. In some embodiments, the second negative pressure level is maintained for 20 minutes or less. In some embodiments, the second negative pressure level is maintained for 5 minutes or less. In some embodiments, the first nitric oxide concentration is maintained for one hour or less. In some embodiments, the first nitric oxide concentration is maintained for 20 minutes or less. In some embodiments, the first nitric oxide concentration is maintained for 5 minutes or less. In some embodiments, the system further comprises one or more indicators configured to indicate a status of the system.

### BRIEF DESCRIPTION OF THE DRAWINGS

The foregoing and other features, aspects, and advantages of the embodiments of the systems, apparatuses, and methods described herein are described in detail below with reference to the drawings of various embodiments, which are intended to illustrate and not to limit the embodiments of the invention. The drawings comprise the following figures in which:

FIGS. 1A-1B illustrate a wound therapy system including a source of negative pressure, a source of nitric oxide, a wound dressing, and a conduit according to some embodiments of this disclosure.

FIG. 1C illustrates certain features of the wound dressing of FIGS. 1A-1B in accordance with some embodiments of this disclosure.

FIG. 1D illustrates a schematic diagram of certain features of the wound therapy system of FIGS. 1A-1B in accordance with some embodiments of this disclosure.

FIG. 2 illustrates a schematic flow diagram of the wound therapy system of FIGS. 1A-1D in accordance with some embodiments of this disclosure.

FIG. 3 illustrates operation of a wound therapy system in accordance with some embodiments of this disclosure.

Throughout the drawings, unless otherwise noted, reference numbers may be re-used to indicate a general correspondence between referenced elements. The drawings are provided to illustrate example embodiments described herein and are not intended to limit the scope of the disclosure.

### DETAILED DESCRIPTION

### Overview

Embodiments disclosed herein relate to systems, devices, and methods for treating a wound. More specifically, embodiments disclosed herein relate generally to systems, devices, and methods for treating a wound with nitric oxide in combination with negative pressure wound therapy (NPWT). In particular, but without limitation, embodiments of this disclosure relate to NPWT and nitric oxide delivery systems and devices, methods for controlling the operation of NPWT and nitric oxide delivery systems and devices, and methods of using NPWT and nitric oxide delivery systems and devices. In some embodiments the systems, devices, and methods described herein advantageously allow for the controlled delivery of nitric oxide such that different concentrations of nitric oxide can be delivered to a wound over the course of treatment. Further, in some embodiments the systems, devices, and methods described herein advantageously allow for the controlled delivery of nitric oxide such that nitric oxide can be delivered to a wound at specific intervals of time over the course of treatment. For example, the systems, devices, and methods described herein can allow for the delivery of a concentration of nitric oxide to a wound that can promote antipathogenic activity for specific intervals of time interspersed with the delivery of a concentration of nitric oxide to the wound that can promote tissue repair and vasodilation that is more sustained over the course of treatment. Further to this example, the application of negative pressure to the wound can facilitate the delivery of nitric oxide to the wound, with such delivery of nitric oxide generally occurring at a reduced pressure so as to provide the therapeutic benefits of both negative pressure and nitric oxide. Embodiments of such systems can generally include a source of nitric oxide, a source of negative pressure, a wound dressing connected to the source of nitric oxide and the source of negative pressure, and associated controller.

The systems, devices, and methods disclosed herein can incorporate or implement any combination of the features described herein. In some cases, the systems, devices, and methods can exclude any of the features described herein. For example, some embodiments disclosed herein can relate to systems, devices, and methods for treating a wound with nitric oxide delivery without NPWT and vice versa. It will be understood by one of skill in the art that application of the systems, devices, and methods described herein are not limited to a particular tissue or a particular wound.

Throughout this specification reference is made to a wound. The term wound is to be broadly construed and encompasses open and closed wounds in which skin is torn, cut or punctured or where trauma causes a contusion, or any other superficial or other conditions or imperfections on the skin of a user or otherwise that benefit from negative pressure treatment and/or treatment with a therapeutic such as nitric oxide. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, bums, diabetic ulcers, pressure ulcers, stoma, surgical wounds, trauma and venous ulcers or the like. In some embodiments, the components of the wound therapy systems described herein can be particularly suited for incisional wounds that exude a small amount of wound exudate.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects pressure that is X mmHg below 760 mmHg or, in other words, a pressure of (760−X) mmHg. In addition, negative pressure that is “less” or “smaller” than X mmHg corresponds to pressure that is closer to atmospheric pressure (e.g., −40 mmHg is less than −60 mmHg). Negative pressure that is “more” or “greater” than-X mmHg corresponds to pressure that is further from atmospheric pressure (e.g., −80 mmHg is more than −60 mmHg). In some embodiments, local ambient atmospheric pressure is used as a reference point, and such local atmospheric pressure may not necessarily be, for example, 760 mmHg.

A healthcare provider, such as a clinician, nurse, or the like, can provide a prescription specifying, for example, the pressure level(s) and/or time(s) of application of negative pressure and/or the concentration(s) and/or time(s) of delivery of nitric oxide. However, the healing process can be different for each user and the prescription may affect the healing process in a way the healthcare provider did not expect at the time of devising the prescription. The healthcare provider may try to adjust the prescription as the wound heals (or does not heal), but such process may require various appointments that can be time consuming and repetitive. Thus, the embodiments disclosed herein provide systems and devices that can allow for efficiently adjusting negative pressure and/or nitric oxide prescriptions and delivering effective negative pressure therapy and/or nitric oxide therapy.

In some embodiments, the wound dressings, wound dressing components, connectors, wound therapy devices, wound therapy systems, and methods described herein can be similar or identical to and/or incorporate any of the features described with respect to any of the wound dressings, connectors, wound therapy devices, wound therapy systems, and/or methods described and/or illustrated in U.S. Pat. No. 9,061,095, titled “WOUND DRESSING AND METHOD OF USE,” issued on Jun. 23, 2015, in U.S. Pat. No. 10,076,594, titled “FLUIDIC CONNECTOR FOR NEGATIVE PRESSURE WOUND THERAPY,” issued on Sep. 18, 2018, and in U.S. Pat. No. 9,084,845, titled “REDUCED PRESSURE THERAPY APPARATUS AND METHODS OF USING SAME,” issued on Jul. 21, 2015, the disclosures of each of which are hereby incorporated by reference in their entirety.

Although the systems, devices, and methods described herein disclose the treatment of a wound with nitric oxide which can provide certain advantages as discussed, such disclosure is not intended to be limiting. The systems, devices, and methods described herein can be implemented with a therapeutic other than nitric oxide or in combination with nitric oxide. For example, therapeutics such as ozone, carbon monoxide, carbon dioxide, oxygen, hydrogen sulfide, and/or others can be used in place of nitric oxide or in combination with nitric oxide or each other. Such therapeutics, including nitric oxide, can preferably be delivered as a gas to the wound, which can facilitate their absorption by the wound and effectiveness. The concentration or concentration range of such therapeutics can be the same, similar, or different than those provided herein for the case of nitric oxide. Furthermore, although the systems, devices, and methods described herein disclose the treatment of a wound with negative pressure in combination with a therapeutic such as nitric oxide, such disclosure is not intended to be limiting. In some embodiments, reference to “in combination” can refer to the serial application of negative pressure and the delivery of a therapeutic. In some embodiments, the systems, devices, and methods described herein can be configured to deliver a therapeutic without application of negative pressure. For example, in some cases a source of negative pressure can be used to evacuate a delivered therapeutic such as nitric oxide from the wound without applying negative pressure wound therapy.

### Nitric Oxide and Negative Pressure Wound Therapy Systems, Devices, and Methods

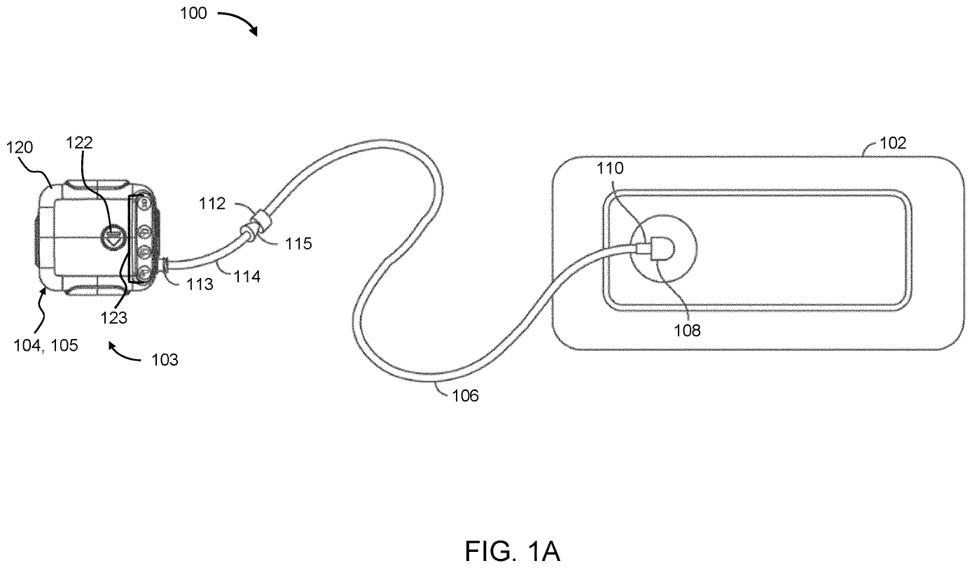

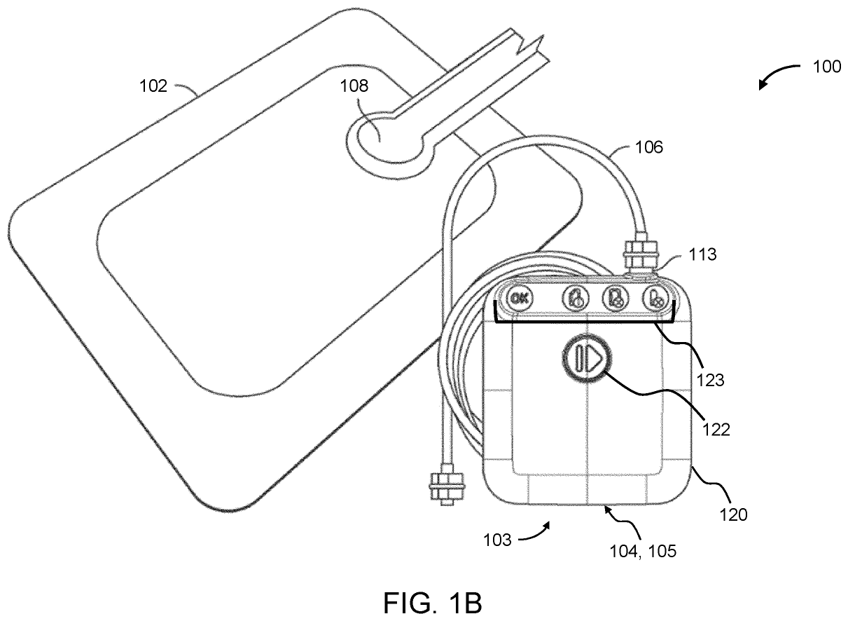

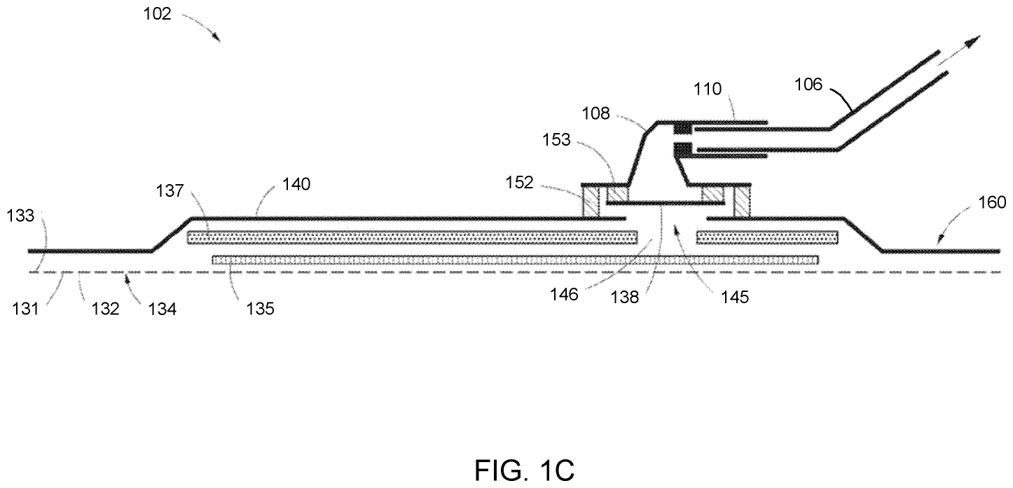

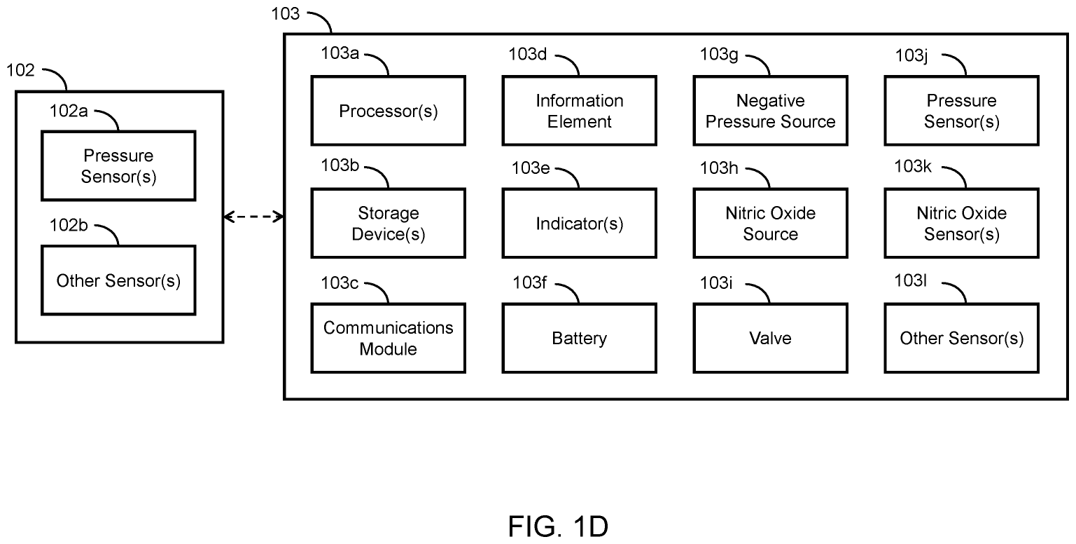

FIGS. 1A and 1B illustrate an embodiment of a wound therapy system 100 configured for application of negative pressure and/or delivery of nitric oxide to a wound. The wound therapy system 100 can comprise a wound dressing 102 (which can also be referred to herein as a “dressing”) in combination with a wound therapy device 103 (which can also be referred to herein as a “wound treatment device”). The wound therapy device 103 can include a source of negative pressure 104 (which can also be referred to herein as a “negative pressure source”) and/or a source of nitric oxide 105 (which can also be referred to herein as a “nitric oxide source”) along with associated controller(s), electrical components, and/or mechanical components. The wound therapy device 103 can include a housing 120 as illustrated configured to house the source of negative pressure 104 and/or the source of nitric oxide 105 along with associated controller(s), electrical components, and/or mechanical components, however in some variants (not shown) the source of negative pressure 104 and the source of nitric oxide 105 can each have their own housing. The wound therapy system 100 can also include a conduit 106 as shown. The wound dressing 102 may be placed over a wound (not illustrated), and the conduit 106 can connect (e.g., fluidly connect) the wound dressing 102 to the source of negative pressure 104 and/or the source of nitric oxide 105 of the wound therapy device 103. The wound dressing 102 or any other wound dressings disclosed herein can be made of any suitable materials, sizes, and components, and can generally include multiple layers such as a superabsorbent layer configured to absorb fluid from the wound and a transmission layer configured to allow transmission of fluid to and away from the wound. In some cases, a wide silicone dressing border, for example, of 3 cm or greater, can be used with the wound dressing 102 to aid in fluidically sealing the wound dressing 102 with the wound and to minimize uncontrolled air leaks into or out of the system. The conduit 106 or any other conduit disclosed herein can be formed from polyurethane, PVC, nylon, polyethylene, silicone, or any other suitable material and can have one or more fluid flow paths (e.g., lumens).

Some embodiments of the wound dressing 102 can have a port 108 configured to receive an end of the conduit 106, though such port 108 is not required. The port 108 can include a connector portion 110 for receiving the conduit 106. In some embodiments, the conduit 106 can otherwise pass through and/or under the wound dressing 102 to apply negative pressure from the negative pressure source 104 and/or deliver nitric oxide from the source of nitric oxide 105 to a space between the wound dressing 102 and the wound so as to maintain a desired level of negative pressure and/or a desired concentration of nitric oxide in such space. Some embodiments of the wound therapy system 100 can be configured such that an end of the conduit 106 is pre-attached to the port 108 of the wound dressing 102 at connector portion 110. The conduit 106 can be any suitable article configured to provide at least a substantially sealed fluid flow pathway between the source of negative pressure 104 and/or the source of nitric oxide 105 and the wound dressing 102, so as to apply the reduced pressure provided by the negative pressure source 104 and/or deliver the nitric oxide provided by the nitric oxide source 105 to the wound covered by the wound dressing 102. In some embodiments, the port 108 can be made of soft, flexible materials such that, for example, a user would experience little or no discomfort if the user lies or otherwise puts pressure on the wound dressing 102 and/or the port 108.

The wound dressing 102 can be provided as a single article with all wound dressing elements (including the port 108) pre-attached and integrated into a single unit. The wound dressing 102 may then be connected, via the conduit 106, to a source of negative pressure and/or a source of nitric oxide such as the negative pressure source 104 and the nitric oxide source 105 of the wound therapy device 103. Wound dressings that may be utilized with the wound therapy devices and system described herein can include Renasys-F, Renasys-G, Renasys AB, and Pico Dressings available from Smith & Nephew. The wound therapy device 103 can include a pump as the source of negative pressure. In some embodiments, though not required, the wound therapy device 103 including the negative pressure source 104 and/or the nitric oxide source 105 can be miniaturized, wearable, and/or portable, similar to the PICO™ pump available from Smith & Nephew, although larger conventional pumps, similar to the EZ CARE™ pump also available from Smith & Nephew, can also be used with the wound dressing 102. The pump can be a diaphragm pump (or any other type of negative pressure pump) actuated by an electric motor, a voice-coil actuator, a piezoelectric actuator, or the like.

In some embodiments, the port 108 can include an enlarged end or head in fluidic communication with the wound dressing 102. The enlarged head can have a round or circular shape as shown. The port 108 can be positioned as illustrated near an edge of the wound dressing 102, but it may also be positioned at any location on the wound dressing 102. For example, some embodiments may provide for a centrally or off-centered location not on or near an edge or corner of the wound dressing 102. In some embodiments, the wound dressing 102 may comprise two or more ports 108, each comprising one or more heads in fluidic communication therewith and one or more connector portion(s) 110.

The wound dressing 102 can be located over a wound site to be treated. The wound dressing 102 can form a substantially sealed cavity or enclosure over the wound site. In some embodiments, it may be preferable for the wound site to be filled partially or completely with a wound packing material. This wound packing material is optional, but may be desirable in certain wounds, for example deeper wounds. The wound packing material can be used in addition to the wound dressing 102. The wound packing material can generally comprise a porous and conformable material, for example foam (including reticulated foams), and gauze. Preferably, the wound packing material is sized or shaped to fit within the wound site so as to fill any empty spaces. The wound dressing 102 can then be placed over the wound site and the wound packing material. When a wound packing material is used, once the wound dressing 102 is sealed over the wound site, negative pressure and/or nitric oxide can be transmitted from the negative pressure source 104 and/or the nitric oxide source 105, respectively, of the wound therapy device 103 through the wound dressing 102, through the wound packing material, and to the wound site. This negative pressure can draw wound exudate and other fluids or secretions away from the wound site.

In some embodiments, the conduit 106 can have a connector 112 positioned at an end opposite the end where it can connect to a wound dressing 102. The connector 112 can be configured to couple with a short length of conduit 114 projecting from the housing 120 of the wound therapy device 103 with a mating connector 115 in communication with the short length of conduit 114, directly to the housing 120 of the wound therapy device 103 with a connector 113, or otherwise. In some embodiments, the connectors 112 and 115 form a quick release connector. The length of the conduit 114 in some embodiments can be approximately 14 mm (0.55 inches), or from approximately 0.5 inches to approximately 5 inches. The short length of conduit 114 can decrease the discomfort to the user while laying or otherwise resting on the housing 120 and connector 112. The short length of conduit 114 can connect at one end directly to the housing 120 of the wound therapy device 103 with connector 113. Configuring the wound therapy device 103 and conduit 106 so that the conduit 106 can be quickly and easily removed from the wound therapy device 103 can facilitate or improve the process of dressing, pump, and/or nitric oxide source changes, if necessary. Any of the wound therapy device 103 embodiments disclosed herein can be configured to have any of the connection configurations disclosed herein between the conduit 106 and its housing 120.

In some embodiments, as in the illustrated embodiment, the wound therapy device 103 comprising the negative pressure source 104 and/or nitric oxide source 105 can be of a sufficiently small and portable size to be supported on the user's body or in the user's clothing or on the wound dressing 102. For example, the wound therapy device 103 can be sized to be attached using adhesive medical tape or otherwise to the user's skin in a comfortable location, adjacent to or on the dressing 102 or otherwise. Further, the wound therapy device 103 can be sized to fit within a pants pocket or a shirt pocket of the user, or can be tethered to the user's body using a lanyard, pouch, or other suitable device.

In any of the embodiments of the wound therapy systems disclosed herein, as in the embodiment illustrated in FIGS. 1A and 1B, the negative pressure source 104 of the wound therapy device 103 can be a canisterless negative pressure source (meaning that the negative pressure source 104 does not have an exudate or liquid collection canister). However, any of the embodiments of the wound therapy systems and devices disclosed herein can be configured to include or support a canister configured to store exudate and/or liquid from the wound. Additionally, in any of the embodiments of the wound therapy systems disclosed herein, any of the negative pressure source and/or nitric oxide source embodiments can be mounted to, embedded within, or supported by the wound dressing 102, or adjacent to the wound dressing 102. In such embodiments the housing 120, portions or the entirety of the conduit 106, the port 108, the connector portion 110, the connector 112, the connector 115, the conduit 114, and/or the connector 113 as shown may be altered or omitted. Further, in some variants (not shown) the dressing 102 can be configured to incorporate the source of negative pressure 104 and/or the source of nitric oxide 105 along with associated controller(s), electrical components, and/or mechanical components, and as such the housing 120, portions or the entirety of the conduit 106, the port 108, the connector 110, the connector 112, the connector 115, the conduit 114, and/or the connector 113 as shown may be altered or omitted.

As mentioned, some embodiments of the wound therapy system 100 are designed to operate without the use of an exudate canister (which can also be referred to herein as a “canister”). The wound dressing 102 can be configured to have a film having a high water vapour permeability to enable the evaporation of surplus fluid, and can have a superabsorbing material contained therein to safely absorb wound exudate. Such a wound dressing 102 can include a filter, such as a hydrophobic filter, that prevents passage of liquids downstream of the wound dressing (toward the negative pressure source 104 and/or nitric oxide source 105). In other embodiments, wound therapy system 100 can be configured to operate with a canister for storing at least part of exudate aspirated from the wound. Such canister can include a filter, such as a hydrophobic filter, that prevents passage of liquids from the wound dressing 102 toward the negative pressure source 104 and/or nitric oxide source 105. In yet other embodiments, both the wound dressing 102 and the canister can include filters that prevent passage of liquids from the wound dressing 102 and the canister towards the negative pressure source 104 and/or nitric oxide source 105. The canister can be included within, a part of, or attached to the wound therapy device 103. In some embodiments, the canister can be a separate component in fluid communication with the wound therapy system 100. Some embodiments of the wound therapy system 100 or components thereof can be designed for single-use therapy and can be disposed of in an environmentally friendly manner after an approximately maximum usage of from three to eleven days. The wound therapy system 100 can be programmed to automatically terminate therapy after a desired number of days, e.g., after seven days, where further operation of the wound therapy system 100 will not be possible. Some embodiments of the wound therapy system 100 or components thereof are designed for longer or repeated usage and/or can be serviceable and can be configured to support and/or include an exudate canister.

FIG. 1C illustrates a cross-sectional view of an embodiment of the wound dressing 102. As shown, a lower surface 131 of the wound dressing 102 can be provided by an optional wound contact layer 132. The wound contact layer 132 can be a polyurethane layer or polyethylene layer or other flexible layer which is perforated, for example via a hot pin process, laser ablation process, ultrasound process or in some other way or otherwise made permeable to liquid and gas. The wound contact layer 132 has a lower surface 131 and an upper surface 133. The perforations 134 are through holes in the wound contact layer which enables fluid to flow through the layer. The wound contact layer helps prevent tissue ingrowth into the other material of the wound dressing. The perforations are small enough to meet this requirement but still allow fluid through. For example, perforations formed as slits or holes having a size ranging from 0.025 mm to 1.2 mm are considered small enough to help prevent tissue ingrowth into the wound dressing while allowing wound exudate to flow into the dressing. The wound contact layer can help hold the whole wound dressing together and help to create an air tight seal around the absorbent pad in order to maintain negative pressure at the wound. The wound contact layer also acts as a carrier for an optional lower and upper adhesive layer (not shown). For example, a lower pressure sensitive adhesive may be provided on the underside surface 131 of the wound dressing whilst an upper pressure sensitive adhesive layer may be provided on the upper surface 133 of the wound contact layer. The pressure sensitive adhesive, which may be a silicone, hot melt, hydrocolloid or acrylic based adhesive or other such adhesives, may be formed on both sides or optionally on a selected one or none of the sides of the wound contact layer. When a lower pressure sensitive adhesive layer is utilized it can help adhere the wound dressing to the skin around a wound site.

A layer 135 of porous material is located above the wound contact layer. This porous layer, or transmission layer, 135 allows transmission of fluid including liquid and gas away from a wound site into upper layers of the wound dressing. In particular, the transmission layer 135 ensures that an open air channel can be maintained to communicate negative pressure and/or nitric oxide over the wound area even when the absorbent layer has absorbed substantial amounts of exudates. The layer should remain open under the typical pressures that will be applied during negative pressure wound therapy as described herein, so that the whole wound site sees an equalized negative pressure and/or an equalized concentration of nitric oxide. The layer 135 can be formed of a material having a three dimensional structure. For example, a knitted or woven spacer fabric (for example Baltex 7970 weft knitted polyester), a non-woven fabric, or other materials could be used.

In some embodiments, the transmission layer 135 comprises a 3D polyester spacer fabric layer including a top layer (that is to say, a layer distal from the wound-bed in use) which is a 84/144 textured polyester, and a bottom layer (that is to say, a layer which lies proximate to the wound bed in use) which is a 100 denier flat polyester and a third layer formed sandwiched between these two layers which is a region defined by a knitted polyester viscose, cellulose or the like monofilament fiber. Other materials and other linear mass densities of fiber could of course be used. Whilst reference is made to a monofilament fiber it will be appreciated that a multistrand alternative could of course be utilized.

The top spacer fabric can have more filaments in a yarn used to form it than the number of filaments making up the yarn used to form the bottom spacer fabric layer. This differential between filament counts in the spaced apart layers helps control moisture flow across the transmission layer. Particularly, by having a filament count greater in the top layer, that is to say, the top layer is made from a yarn having more filaments than the yarn used in the bottom layer, liquid tends to be wicked along the top layer more than the bottom layer. In use, this differential tends to draw liquid away from the wound bed and into a central region of the dressing where the absorbent layer helps lock the liquid away or itself wicks the liquid onwards towards the cover layer where it can be transpired.

A layer 137 of absorbent material is provided above the transmission layer 135. The absorbent material which may be a foam or non-woven natural or synthetic material and which may optionally include or be super-absorbent material forms a reservoir for fluid, particularly liquid, removed from the wound site and draws those fluids towards a cover layer 140. The material of the absorbent layer also prevents liquid collected in the wound dressing from flowing in a sloshing manner. The absorbent layer 137 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 137 may typically be manufactured from ALLEVYN™ foam, Freudenberg 114-224-4 and/or Chem-Posite™ 11C-450.

In some embodiments, the absorbent layer 137 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand −like fibers leads to strong capillary action in the fibrous pad which helps distribute liquid. In this way, the super-absorbent material can be efficiently supplied with liquid. Also, all regions of the absorbent layer can be provided with liquid. The wicking action can also assist in bringing liquid into contact with the upper cover layer to increase transpiration rates of the dressing. The wicking action can also assist in delivering liquid downwards towards the wound bed when exudation slows or halts. This delivery process can help maintain the transmission layer and lower wound bed region in a moist state which helps prevent crusting within the dressing (which could lead to blockage) and helps maintain an environment optimized for wound healing.

In some embodiments, the absorbent layer 137 may be an air-laid material. Heat fusible fibers may optionally be used to assist in holding the structure of the pad together. It will be appreciated that rather than using super-absorbing particles or in addition to such use, the absorbent layer may include synthetic stable fibers and/or bi-component stable fibers and/or natural stable fibers and/or super-absorbent fibers. An example of a suitable material is the Product Chem-Posite™ 11 C available from Emerging Technologies Inc (ETi) in the USA. In some embodiments, the absorbent layer 137 is formed by fibers which operate to lock super-absorbent particles within the absorbent layer. This helps ensure that super-absorbent particles do not move external to the absorbent layer and towards an underlying wound bed.

The absorbent layer 137 may comprise a layer of multiple fibers. Preferably, the fibers are strand −like and made from cellulose, polyester, viscose or the like. Preferably, dry absorbent particles are distributed throughout the absorbent layer ready for use. In some embodiments, the absorbent layer 137 comprises a pad of cellulose fibers and a plurality of super absorbent particles. In additional embodiments, the absorbent layer is a non-woven layer of randomly orientated cellulose fibers.

Super-absorber particles/fibers may be, for example, sodium polyacrylate or carbomethoxycellulose materials or the like or any material capable of absorbing many times its own weight in liquid. In some embodiments, the material can absorb more than five times its own weight of 0.9% W/W saline, etc. In some embodiments, the material can absorb more than 15 times its own weight of 0.9% W/W saline, etc. In some embodiments, the material is capable of absorbing more than 20 times its own weight of 0.9% W/W saline, etc. Preferably, the material is capable of absorbing more than 30 times its own weight of 0.9% W/W saline, etc.

Preferably, the particles of superabsorber are very hydrophilic and grab the fluid as it enters the dressing, swelling up on contact. An equilibrium is set up within the dressing core whereby moisture passes from the superabsorber into the dryer surrounding area and as it hits the top film the film switches and the fluid vapor starts to be transpired. A moisture gradient is established within the dressing to continually remove fluid from the wound bed and ensure the dressing does not become heavy with exudate.

Preferably the absorbent layer 137 includes at least one through-hole located so as to underly the suction port. As illustrated in FIG. 1C a single through-hole 146 can be used to produce an opening underlying the port 108. It will be appreciated that multiple openings could alternatively be utilized. Additionally should more than one port be utilized according to certain embodiments one or multiple openings may be made in the super-absorbent layer in registration with each respective port. Although not essential to certain embodiments the use of through holes in the super-absorbent layer provide a fluid flow pathway which is particularly unhindered and this is useful in certain circumstances.

Where a through-hole 146 is provided in the absorbent layer 137 the thickness of the layer itself will act as a stand −off separating any overlying layer from the upper surface (that is to say the surface facing away from a wound in use) of the transmission layer 135. An advantage of this is that a filter of the port is thus decoupled from the material of the transmission layer. This helps reduce the likelihood that the filter will be wetted out and occlude and/or block further operation.

Use of one or more through holes in the absorbent layer 137 also has the advantage that during use if the absorbent layer contains a gel forming material, such as superabsorber, that material as it expands to absorb liquid, does not form a barrier through which further liquid movement and fluid movement in general cannot pass. In this way each opening in the absorbent layer provides a fluid pathway between the transmission layer 135 directly to the wound facing surface of the filter and then onwards into the interior of the port 108.

A gas impermeable, but moisture vapor permeable, cover layer 140 extends across the width of the wound dressing. The cover layer, which may for example be a polyurethane film (for example, Elastollan SP9109) having a pressure sensitive adhesive on one side, is impermeable to gas and this layer thus operates to cover the wound and to seal a wound cavity over which the wound dressing 102 is placed. In this way an effective chamber is made between the cover layer and a wound site where a negative pressure can be established. The cover layer 140 is sealed to the wound contact layer 132 in a border region 160 around the circumference of the dressing, ensuring that no air is drawn in through the border area, for example via adhesive or welding techniques. The cover layer 140 protects the wound from external bacterial contamination (bacterial barrier) and allows liquid from wound exudates to be transferred through the layer and evaporated from the film outer surface. The cover layer 140 typically comprises two layers; a polyurethane film and an adhesive pattern spread onto the film. The polyurethane film is moisture vapor permeable and may be manufactured from a material that has an increased water transmission rate when wet.

The absorbent layer 137 may be of a greater area than the transmission layer 135, such that the absorbent layer 137 overlaps the edges of the transmission layer 135, thereby ensuring that the transmission layer 135 does not contact the cover layer 140. This provides an outer channel of the absorbent layer 137 that is in direct contact with the wound contact layer 132, which aids more rapid absorption of exudates to the absorbent layer 137. Furthermore, this outer channel ensures that no liquid is able to pool around the circumference of the wound cavity, which may otherwise seep through the seal around the perimeter of the dressing leading to the formation of leaks.

The absorbent layer 137 is positioned in fluid contact with the cover layer 140. As the absorbent layer 137 absorbs wound exudate, the exudate is drawn towards the cover layer 140, bringing the water component of the exudate into contact with the moisture vapor permeable cover layer. This water component is drawn into the cover layer itself and then evaporates from the top surface of the dressing. In this way, the water content of the wound exudate can be transpired from the dressing, reducing the volume of the remaining wound exudate that is to be absorbed by the absorbent layer 137, and increasing the time before the dressing becomes full and must be changed. This process of transpiration occurs even when negative pressure has been applied to the wound cavity, and it has been found that the pressure difference across the cover layer when a negative pressure is applied to the wound cavity has negligible impact on the moisture vapor transmission rate across the cover layer.

An orifice 145 is provided in the cover film 140 to allow a negative pressure to be applied to the wound dressing 102. The port 108 is sealed to the top of the cover film 140 over the orifice 145, and communicates negative pressure and/or nitric oxide through the orifice 145. The conduit 106 may be coupled at one end to the port 108 at connector portion 110 and at another end to the wound therapy device 103 (not shown) as discussed herein to allow fluids to be pumped out of the dressing and/or for the delivery of nitric oxide. The port 108 may be adhered and sealed to the cover film 140 using an adhesive such as an acrylic, cyanoacrylate, epoxy, UV curable or hot melt adhesive. The port 108 can be formed from a soft polymer, for example a polyethylene, a polyvinyl chloride, a silicone or polyurethane having a hardness of 30 to 90 on the Shore A scale.

An aperture or through-hole 146 is provided in the absorbent layer 137 beneath the orifice 145 such that the orifice is connected directly to the transmission layer 135. This allows the negative pressure applied and/or nitric oxide delivered to the port 108 to be communicated to the transmission layer 135 without passing through the absorbent layer 137. This can ensure that the negative pressure applied and/or nitric oxide delivered to the wound site is not inhibited by the absorbent layer as it absorbs wound exudates. In some embodiments, no aperture may be provided in the absorbent layer 137, or alternatively a plurality of apertures underlying the orifice 145 may be provided.

A filter element 138 that is impermeable to liquids, but permeable to gases can be provided to act as a liquid barrier, and to ensure that no liquids are able to escape from the wound dressing. The filter element 138 may also function as a bacterial barrier. Typically the pore size is 0.2 μm. Suitable materials for the filter material of the filter element 138 include 0.2 micron Gore™ expanded PTFE from the MMT range, PALL Versapore™ 200R, and Donaldson™ TX6628. Larger pore sizes can also be used but these may require a secondary filter layer to ensure full bioburden containment. As wound fluid contains lipids it is preferable, though not essential, to use an oleophobic filter membrane for example 1.0 micron MMT-332 prior to 0.2 micron MMT-323. This prevents the lipids from blocking the hydrophobic filter. The filter element can be attached or sealed to the port 108 and/or the cover film 140 over the orifice 145. For example, the filter element 130 may be molded into the port 108, or may be adhered to both the top of the cover layer 140 and bottom of the port 108 using an adhesive such as, but not limited to, a UV cured adhesive. The filter element 138 thus enables gas to flow through the orifice 145 while containing liquid, particulates and pathogens in the wound dressing 102.

It will be understood that other types of material could be used for the filter element 138. More generally a microporous membrane can be used which is a thin, flat sheet of polymeric material, this contains billions of microscopic pores. Depending upon the membrane chosen these pores can range in size from 0.01 to more than 10 micrometers. Microporous membranes are available in both hydrophilic (water filtering) and hydrophobic (water repellent) forms. In some embodiments, filter element 138 comprises a support layer and an acrylic co-polymer membrane formed on the support layer. Preferably the wound dressing 102 according to certain embodiments uses microporous hydrophobic membranes (MHMs). Numerous polymers may be employed to form MHMs. For example, PTFE, polypropylene, PVDF and acrylic copolymer. All of these optional polymers can be treated in order to obtain specific surface characteristics that can be both hydrophobic and oleophobic. As such these will repel liquids with low surface tensions such as multi-vitamin infusions, lipids, surfactants, oils and organic solvents. MHMs block liquids whilst allowing air to flow through the membranes. They are also highly efficient air filters eliminating potentially infectious aerosols and particles. The filter element 138 may also include an odor absorbent material, for example activated charcoal, carbon fiber cloth or Vitec Carbotec-RT Q2003073 foam, or the like. For example, an odor absorbent material may form a layer of the filter element 138 or may be sandwiched between microporous hydrophobic membranes within the filter element.

The wound dressing 102 can include spacer elements 152, 153 in conjunction with the port 108 and the filter 138. With the addition of such spacer elements 152, 153, the port 108 and filter 138 may be supported out of direct contact with the absorbent layer 137 and/or the transmission layer 135. The absorbent layer 137 may also act as an additional spacer element to keep the filter 138 from contacting the transmission layer 135. Accordingly, with such a configuration contact of the filter 138 with the transmission layer 135 and wound fluids during use may thus be minimized. The aperture 146 through the absorbent layer 137 can be as large or larger than the port 108, or may only need to be large enough such that an air path can be maintained from the port 108 to the transmission layer 135 when the absorbent layer 137 is saturated with wound fluids.

In particular for embodiments with a single port 108 and through-hole 146, it may be preferable for the port 108 and through-hole 146 to be located in an off-center position as illustrated in FIGS. 1A-1C. Such a location may permit the wound dressing 102 to be positioned onto a patient such that the port 108 is raised in relation to the remainder of the dressing 102. So positioned, the port 108 and the filter 138 may be less likely to come into contact with wound fluids that could prematurely occlude the filter 138 so as to impair the transmission of negative pressure and/or nitric oxide to the wound site.

FIG. 1D illustrates a schematic diagram of certain features which can be incorporated in the wound therapy system 100 as well as any embodiments of wound therapy system(s) described herein. As shown, the wound therapy device 103 can include one or more processors 103a, one or more storage devices 103b, a communication module 103c, an information element 103d, one or more indicators 103e, a battery 103f, a negative pressure source 103g, a nitric oxide source 103h, a valve 103i, one or more pressure sensors 103j, one or more nitric oxide sensors 103k, and one or more other sensors 103l. The wound dressing 102 (or in some embodiments, the conduit 106) can include one or more pressure sensors 102a and one or more other sensors 102b.

The processor(s) 103a can be configured, among other things, to process data, execute instructions and/or be programmed to perform one or more functions, and/or control the operation of the wound therapy system 100 (and as such, the processor(s) 103a can also be referred to herein as a “controller”). For example, the processor(s) 103a can control operation of the negative pressure source 103g (which can correspond to the negative pressure source 104 discussed herein) and/or the nitric oxide source 103h (which can correspond to the nitric oxide source 105 discussed herein). In some embodiments, the processor(s) 103a can control operation of the negative pressure source 103g by controlling operation of a pump as discussed herein. In some embodiments, the processor(s) 103a can control operation of the nitric oxide source 103h by controlling operation of the valve 103i. As another example, the processor(s) 103a can process signals and/or data received and/or obtained by sensor(s) of the wound therapy system 100, such as the pressure sensor(s) 103k and/or 102a, the nitric oxide sensor(s) 103k, and/or the other sensor(s) 103l and/or 102b. The processor(s) 103a can utilize such signals/data from the sensor(s) to determine a status of the system, and in some circumstances provide an alert and/or alarm to the user based on the status of the system via indicator(s) 103e. Further, the processor(s) 103a can execute instructions to perform functions related to storing, transmitting, and/or receiving data. The processor(s) 103a can also operate to accept user input and provide output to the user.

The processor(s) 103a can include a general purpose controller, such as a low-power processor, or one or more application specific controllers. The processor(s) 103a can be configured with one processor as a “central” processor in the electronic architecture of the control system with it coordinating the activity of other processors. In some embodiments, the processor(s) 103a can include a user interface processor, a communications processor, a negative pressure control processor (e.g., a pump control processor), a nitric oxide source control processor, and/or one or more other processors. The processor(s) 103a can run a suitable operating system, such as a Linux, Windows CE, VxWorks, or similar.

The storage device(s) 103b can include one or more memory devices that store data, including without limitation, dynamic and/or static random access memory (RAM), programmable read-only memory (PROM), erasable programmable read-only memory (EPROM), electrically erasable programmable read-only memory (EEPROM), magnetic memory, solid-state memory, Magnetoresistive random-access memory (MRAM), and the like. Such stored data can be processed and/or unprocessed data obtained from the wound therapy system 100, GPS data, therapy data, device data, and event data. This data can also include user data collected by one or more sensors as described herein. The processor(s) 103a can track and log therapy and other operational data, which can also be stored in memory of the storage device(s) 103b. In some embodiments, the storage device(s) 103b can also include external data sources such as one or more expansion modules, one or more USB ports, SD ports, Compact Disc (CD) drives, DVD drives, FireWire ports, Thunderbolt ports, PCI Express ports, and the like. The processor(s) 103a can store data in memory of the storage device(s) 103b, which can be internal or external to the processor(s) 103a.

The communication module 103c can facilitate communication (via wires and/or wireless connection) between the wound therapy system 100 (and/or components thereof) and separate devices, such as separate monitoring, computing, electrical, and/or mobile devices. The communication module 103c can include one or more transceivers (such as a wireless transceiver) for sending and receiving data. Such a transceiver can include one or more antennas, optical sensors, optical transmitters, vibration motors or transducers, vibration sensors, acoustic sensors, ultrasound sensors, or the like. The communication module 103c can be configured to allow the wound therapy system 100 to communicate via wires or wirelessly with other devices, systems, and/or networks over any of a variety of communication protocols. The communication module 103c can provide one or more of the following types of connections: Global Positioning System (GPS), cellular connectivity (for example, 2G, 3G, LTE, 4G, 5G, or the like), near field communication (NFC), Bluetooth connectivity, radio frequency identification (RFID), wireless local area network (WLAN), wireless personal area network (WPAN), WiFi connectivity, Internet connectivity, optical connectivity (for example, using infrared light, barcodes, such as QR codes, etc.), acoustic connectivity, ultrasound connectivity, satellite transmission, proprietary protocols, combinations of the same, and the like. Connectivity can be used for various activities, such as location tracking, asset tracking, compliance monitoring, remote selection, uploading of logs, alarms, and other operational data, and adjustment of therapy settings, upgrading of software or firmware, pairing, and the like. The communication module 103c can communicate information to the processor(s) 103a. The communications module 103c can include internal memory or can utilize memory of storage device(s) 103b.

Using the connectivity provided by the communication module 103c, the wound therapy system 100 can upload any of the data stored, maintained, or tracked to a remote computing device. The wound therapy system 100 can also download various operational data, such as therapy selection and parameters, firmware and software patches and upgrades, and the like, via the communication module 103c. The communication module 103c can also facilitate communication between components of the wound therapy system 100. As such, the communication module 103c can include an input/output module. The sensors of the wound therapy system 100 can be connected to the input/output module of the communication module 103c and the processor(s) 103a. The input/output module can receive data from the sensors of the system through one or more ports, such as serial (for example, I2C), parallel, hybrid ports, and the like.

The information element 103d can be a memory storage element that stores, in non-volatile memory, information used to help maintain a standard of quality associated with the wound therapy system 100. Illustratively, the information element 103d can store information regarding whether the wound therapy system 100 has been previously activated and whether the wound therapy system 100 has been previously operational for a prolonged period of time, such as, for example, four hours, one day, two days, three days, four days, five days, seven days, eleven days, or any period of time. The information stored in the information element 103d can be used to help detect improper re-use of the wound therapy system 100, for example.

The indicator(s) 103e can be configured to provide indications, alarms, and/or the like to the user and/or their health care provider reflecting operating or failure conditions of the wound therapy system 100 and/or any of its components, such as the negative pressure source 104 and/or the nitric oxide source 105 of the wound therapy device 103. The wound therapy system 100 can include visual, audible, tactile, and other types of indicator(s) 103e configured to alert and/or alarm the user of various operating and/or failure conditions and/or to provide feedback to the user. Such conditions can include system on/off, standby, pause, normal operation, negative pressure application, delivery of nitric oxide, pump failure, power supply failure, the remaining capacity or life of a power source (e.g., such as voltage level of a battery), dressing problem, leak detected, suction blockage (e.g., to the source of negative pressure), the fluid capacity and/or remaining fluid capacity of a dressing, error, the capacity and/or remaining capacity of the nitric oxide source, nitric oxide source low, nitric oxide source depletion, the capacity and/or remaining capacity of a canister (if equipped), and any other similar or suitable conditions or combinations thereof. In some embodiments, the wound therapy device 103 can include such one or more indicator(s) 103e. The indicator(s) 103e can include one or more speakers, displays, light sources, vibration motors, and/or combinations thereof. The processor(s) 103a can be in communication with the indicator(s) 103e and can be configured to instruct the indicator(s) 103e to alert and/or alarm. In some embodiments, the processor(s) 103a can provide instruction to the indicator(s) 103e to provide an alert and/or an alarm responsive to a condition of the wound therapy system 100 and/or any components thereof. Alternatively, or in addition, indication can be provided by activating or deactivating a negative pressure source 103g (such as negative pressure source 104 disclosed herein), reducing the negative pressure level generated by the negative pressure source 103g, lowering the amount of power used by the negative pressure source 103g, or any combination thereof.

An implementation of indicator(s) 103e can be indicators 123 shown in FIGS. 1A-1B. As shown, indicators 123 can be one or more light emitting diodes (LEDs) and/or icons included with the housing 120 of the wound therapy device 103. The indicators 123 can be positioned on the housing 120 of the wound therapy device 103 and can be configured to alert and/or alarm the user to a variety of operating and/or failure conditions of the wound therapy system 100, including those listed above. An exemplary set of indicators 123 can include an “OK” indicator which can indicate normal operation of the wound therapy system 100, a “leak” indicator which can indicate the existence of a leak in the wound therapy system 100 or components thereof, a “dressing full” indicator which can indicate that a wound dressing is at or near capacity, a “battery critical” indicator which can indicate that the battery is at or near a critical level, and/or a “therapy delivered” indicator which can indicate that nitric oxide and/or negative therapy is being delivered to the wound dressing. In some embodiments, the indicators can have a green and/or orange color, and/or can be illuminated with a green and/or orange light (e.g., colored LEDs, although any color/wavelength(s) of light may be used).

In some embodiments the wound therapy system 100, in particular the housing 120 of the wound therapy device 103, can include an indicator in the form of a display configured to provide the user with information (e.g., information regarding an operational status of the wound therapy system 100). The display (not shown) can be a touch screen display. The display can support playback of audiovisual (AV) content, such as instructional videos, and render a number of screens or graphical user interfaces (GUIs) for configuring, controlling, and monitoring the operation of the wound therapy device 103. The display can also include one or more icons which can alert and/or alarm the user of one or more operating and/or failure conditions of the wound therapy system 100 and/or any component thereof, such as those listed above. In some embodiments, the display can include icons that are similar to the indicators 123 described above. An exemplary set of icons can include an “OK” icon which can indicate normal operation of the wound therapy system 100, a “leak” icon which can indicate the existence of a leak in the wound therapy system 100 or components thereof, a “dressing full” icon which can indicate that a wound dressing is at or near capacity, a “battery critical” icon which can indicate that the battery is at or near a critical level, and/or a “therapy delivered” icon which can indicate that nitric oxide and/or negative therapy is being delivered to the wound dressing.

In some embodiments, the wound therapy system 100, in particular the housing 120 of the wound therapy device 103, can include one or more user input features, such as control button 122, designed to receive an input from the user for controlling the operation of the wound therapy system 100. In the embodiment shown in FIGS. 1A-1B, a single control button 122 is present which can be used to activate and deactivate the wound therapy system 100 and/or control other operating parameters of the wound therapy system 100. For example, in some embodiments, the control button 122 can be used to activate the application of negative pressure, pause the application of negative pressure, activate the delivery of nitric oxide, pause the delivery of nitric oxide, clear indicators such as indicators 123 or any icons on a display, power on/off the wound therapy device 103, and/or be used for any other suitable purpose for controlling an operation of the wound therapy system 100 (e.g., by sequentially pushing on the button 122). The button 122 can be a push style button that can be positioned on an outside, front surface of the housing 120 of the wound therapy device 103. In some variants (not shown), multiple input features (e.g., multiple buttons) can be provided on the housing 120 of the wound therapy device 103 for controlling any operation of the wound therapy system 100.

In some embodiments, the wound therapy system 100, in particular the housing 120 of the wound therapy device 103, can include one or more speakers for producing sound. The speaker(s) can generate an acoustic alarm in response to deviations in therapy delivery, non-compliance with therapy delivery, or any other similar or suitable conditions listed above or any combinations thereof.

The battery 103f can provide power for hardware components of the wound therapy system 100 described herein. The battery 103f can comprise one or more batteries. The battery 103f can be non-rechargeable or rechargeable. For example, the battery 102d can be a Zinc-Air, lithium, a lithium polymer, a lithium-ion, a lithium-ion polymer, a lead-acid, a nickel-cadmium, or a nickel-metal hydride battery. Additionally or alternatively, the wound therapy system 100, in particular the wound therapy device 103, can be configured to obtain power from a power source that is external to the wound therapy system 100. For example, the wound therapy device 103 can include or can be configured to connect to a cable which can itself connect to an external power source to provide power to the wound therapy device 103.

The negative pressure source 103g can be configured to generate negative or reduced pressure for application to a wound dressing. In some embodiments, the negative pressure source 103g includes a pump as its source of negative pressure. The pump can be a suitable pump, such as a diaphragm pump, peristaltic pump, rotary pump, rotary vane pump, scroll pump, screw pump, liquid ring pump, diaphragm pump operated by a piezoelectric transducer, voice coil pump, and the like. The processor(s) 103a (e.g., controller) can be in communication with the negative pressure source 103g and control/regulate its operation. For example, the processor(s) 103a in cooperation with the negative pressure source 103g can measure pressure in a fluid flow path, using data received from pressure sensor(s) 103j and/or 102a, calculate the rate of fluid flow, and control the pump. The processor(s) 103a can control the pump motor so that a desired level of negative pressure in achieved at the wound site. The desired level of negative pressure can be pressure set, selected by the user, or as otherwise disclosed herein. The processor(s) 103a can control the pump (for example, pump motor) using pulse-width modulation (PWM). A control signal for driving the pump can be a 0-100% duty cycle PWM signal.

An implementation of the negative pressure source 103g can be negative pressure source 104 discussed herein. The negative pressure source 104, and thus the wound therapy device 103, can be configured to apply negative pressure of approximately −80 mmHg, or between about −20 mmHg and −200 mmHg to the wound dressing 102. Note that these pressures are relative to normal ambient atmospheric pressure thus, −200 mmHg would be about 560 mmHg in practical terms. In some cases, the pressure range can be between about −40 mmHg and −150 mmHg. Alternatively, a pressure range of up to −75 mmHg, up to −80 mmHg or over −80 mmHg can be used. Also in some cases a pressure range of below −75 mmHg can be used. Alternatively, a pressure range of over approximately −100 mmHg, or even −150 mmHg, can be supplied by the wound therapy device 103.

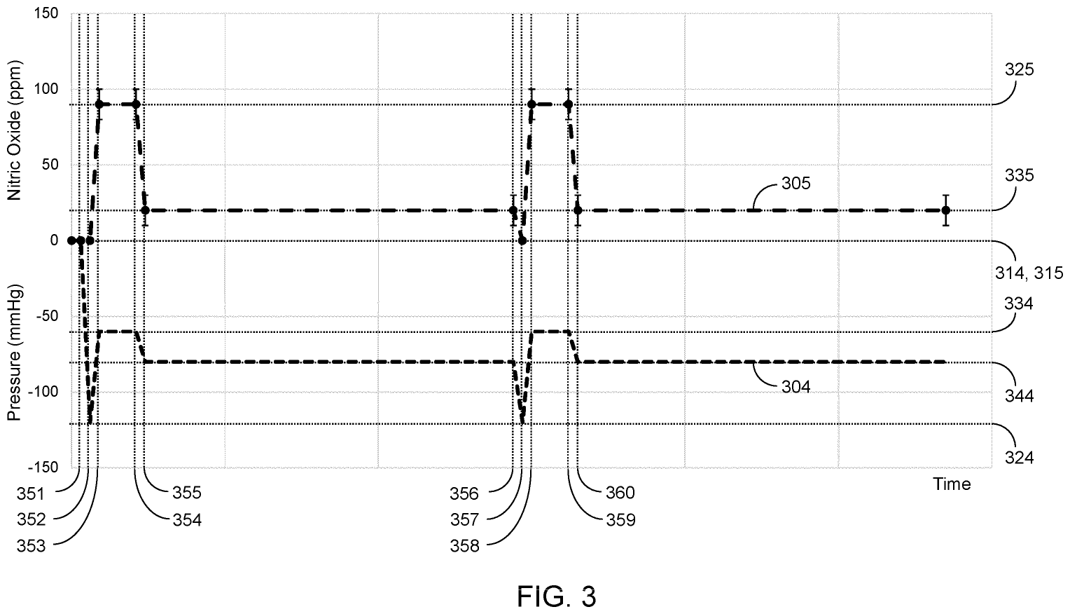

The negative pressure source 104, and thus the wound therapy device 103, can be configured to apply continuous or intermittent negative pressure therapy to the wound dressing 102. Continuous negative pressure therapy can be applied at negative pressures greater than 0 mmHg, −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, −200 mmHg, or less than −200 mmHg. Intermittent negative pressure therapy can be applied between low and high negative pressure set points (sometimes referred to as “set point” or “pressure level”). Low set point can be set at or above (with “above” meaning greater negative pressure than) 0 mmHg, −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, or at or below −180 mmHg. High set point can be set at or above −25 mmHg, −40 mmHg, −50 mmHg, −60 mmHg, −70 mmHg, −80 mmHg, −90 mmHg, −100 mmHg, −120 mmHg, −140 mmHg, −160 mmHg, −180 mmHg, −200 mmHg, or below −200 mmHg. During intermittent therapy, negative pressure at low set point can be applied for a first time duration, and upon expiration of the first time duration, negative pressure at high set point can be applied for a second time duration. Upon expiration of the second time duration, negative pressure at low set point can be applied. The first and second time durations can be same or different values. In some cases, the negative pressure source 104 can apply intermittent negative pressure therapy at a first negative pressure level, a second negative pressure level, and a third negative pressure level. The third negative pressure level can be between the first and second negative pressure levels. The first negative pressure level can include a pressure range of between about −80 mmHg and about −200 mmHg. The second negative pressure level can include a pressure range of between about −20 mmHg and about −120 mmHg. The third negative pressure level can include a pressure range of between about −40 mmHg and about −200 mmHg. Such intermittent negative pressure therapy can be applied in conjunction with delivery of nitric oxide to the wound dressing 102 from nitric oxide source 105, which will be described in further detail with respect to FIG. 3.

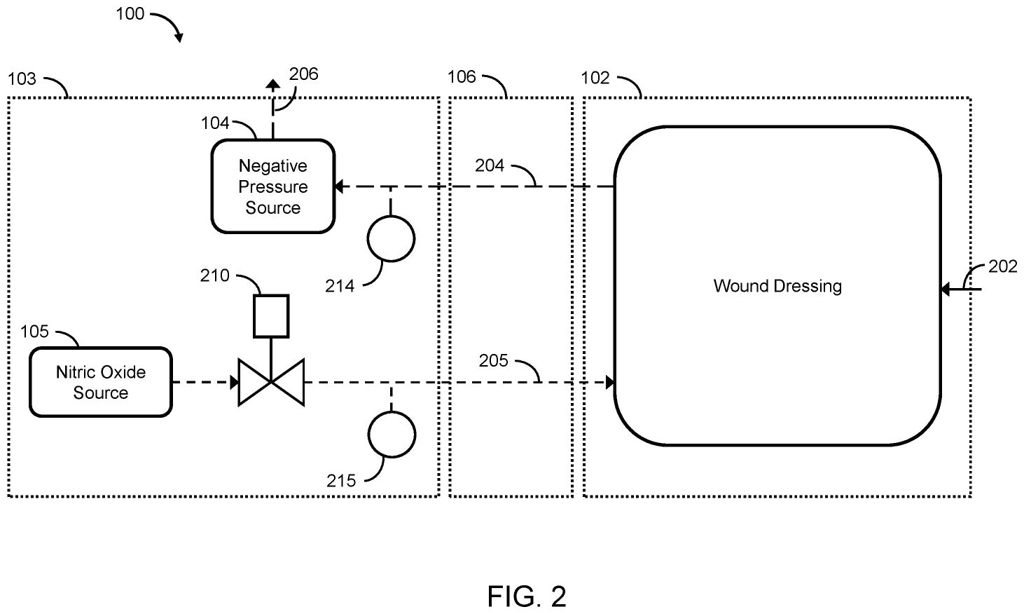

The nitric oxide source 103h can be configured to deliver nitric oxide to a wound dressing. In some embodiments, the nitric oxide source 103h includes one or more pressurized cartridges of nitric oxide gas as its source of nitric oxide. Such cartridge(s) can be any cartridge configured to releasably connect (e.g., screw fit, push fit, or clip into place) to components of the wound therapy device 103 and/or the fluid flow pathway to the wound dressing 102. In some cases, such cartridge(s) of nitric oxide can be provided with the wound therapy device 103. In some embodiments, such cartridge(s) of nitric oxide can be replaced by the user as needed. Such cartridge(s) can be of about the same pressure and concentration of nitric oxide, or they may be different. As an example, the cartridge(s) can be configured to contain approximately 20 cubic centimeters of nitric oxide at approximately 900 psi. In some cases, the cartridge(s) can be less than approximately 4 inches long and less than approximately 1 inch in diameter. In embodiments with the nitric oxide source 103h including one or more pressurized cartridges of nitric oxide gas as its source of nitric oxide, a valve 103i can be configured to regulate the delivery of the nitric oxide from such cartridge(s). The valve 103i can be in electrical communication with the processor(s) 103a and the processor(s) 103a can operate the valve 103i to regulate nitric oxide delivery to the wound dressing 102. The valve 103i can be a low power active valve, for example, a solenoid valve. An implementation of the valve 103i can be a valve 210 described with respect to FIG. 2 herein.

In some embodiments, the nitric oxide source 103h includes a nitric oxide generator as its source of nitric oxide. Such nitric oxide generator can include a plasma generator that can produce nitric oxide from air and/or an oxygen rich gas mixture. As an example, the nitric oxide generator can be similar to and/or incorporate features of a handheld plasma pen that can generate nitric oxide from air via a high-voltage source. The nitric oxide generator can connect to components of the wound therapy device 103 and/or the fluid flow pathway to the wound dressing 102. The nitric oxide generator can be in electrical communication with the processor(s) 103a and the processor(s) 103a can operate the nitric oxide generator to regulate nitric oxide delivery to the wound dressing 102. The nitric oxide generator can be configured for low power consumption. In embodiments with the nitric oxide source 103h including a plasma generator, the valve 103i can be optional or not required. In some implementations, the delivery of nitric oxide from the nitric oxide source 103h can be manually controlled by the user and/or their healthcare provider.

An implementation of the nitric oxide source 103h can be nitric oxide source 105 discussed herein. The nitric oxide source 105, and thus the wound therapy device 103, can be configured to deliver nitric oxide in a concentration of between about 0 parts per million (ppm) and about 500 ppm to the wound dressing 102. In some cases, the nitric oxide source 105 can deliver a concentration of 5 ppm or greater to the wound dressing 102. In certain examples, the nitric oxide concentration can range between about: 50 ppm to 450 ppm, 100 ppm to 400 ppm, 150 ppm to 350 ppm, 200 ppm to 300 ppm, or about 250 ppm. In some embodiments, the nitric oxide source 105 can deliver a concentration of nitric oxide to the wound dressing 102 such that tissue of the wound reaches a nitric oxide concentration of between about 0 nanomolar (nM) and about 2000 nM. In some cases, the nitric oxide source 105 can advantageously be configured to deliver nitric oxide at different concentrations, such as at a concentration to promote antipathogenic activity (which can also be referred to herein as a “high concentration”) and at a concentration to promote tissue repair and vasodilation (which can also be referred to herein as a “low concentration”). The concentration of nitric oxide to promote antipathogenic activity can be between about 80 ppm and about 200 ppm. In some cases, the concentration of nitric oxide to promote antipathogenic activity can be such that tissue of the wound reaches a nitric oxide concentration between about 100 nM and about 2000 nM. The concentration of nitric oxide to promote tissue repair and vasodilation activity can be between about 0 ppm and about 80 ppm. In some cases, the concentration of nitric oxide to promote tissue repair and vasodilation activity can be such that tissue of the wound reaches a nitric oxide concentration between about 0 nM and about 100 nM. Examples of tissue repair and vasodilation activities can include fibroblast cell migration, keratinocyte differentiation, macrophage polarization, and endothelial cell migration. The concentration of nitric oxide to promote tissue repair and vasodilation activity can be between a factor of 5 and a factor of 15 less than the concentration of nitric oxide to promote antipathogenic activity. In some cases, the concentration of nitric oxide to promote tissue repair and vasodilation activity can be about a factor of 10 less than the concentration of nitric oxide to promote antipathogenic activity. In certain examples, the wound tissue may reach a concentration of about: 200 nM to 1800 nM, 400 nM to 1600 nM, 600 nM to 1400 nM, 800 nM to 1200 nM, or about 1000 nM. The delivery of different concentrations of nitric oxide can be done at different times and/or for different periods of time. For example, the nitric oxide source 105 can deliver nitric oxide to the wound dressing 102 at the high concentration for less than or about 1 minute, less than or about 2 minutes, less than or about 5 minutes, less than or about 10 minutes, less than or about 15 minutes, less than or about 20 minutes, less than or about 1 hour, less than or about 2 hours, or greater than about 2 hours. Such delivery of nitric oxide can be performed serially and/or intermittently. The nitric oxide source 105 can deliver nitric oxide to the wound dressing 102 at the low concentration for greater than or about 1 minute, greater than or about 5 minutes, greater than or about 10 minutes, greater than or about 20 minutes, greater than or about 30 minutes, greater than or about 1 hour, greater than or about 2 hours, greater than or about 5 hours, continuously, or nearly continuously except for periods of time wherein the high dose of nitric oxide is delivered or when negative pressure is applied to the wound dressing 102 or when therapy is paused or ceased by the user. The nitric oxide source 105 can be configured to alternate the delivery of the high dose of nitric oxide with the low dose of nitric oxide. In some cases, nitric oxide can be delivered to the wound dressing 102 in conjunction with application of negative pressure from the negative pressure source 104, which will be described in further detail with respect to FIG. 3. In some embodiments, the user and/or their healthcare provider can interact with the wound therapy system 100 to input and/or select the concentration(s) of nitric oxide to be delivered to the wound dressing 102 and/or the timing of nitric oxide delivery.

The pressure sensor(s) 103j of the wound therapy device 103 and the pressure sensor(s) 102a of the wound dressing 102 can include one or more pressure sensors configured to measure a pressure level within the wound therapy system 100 and/or aspects thereof. The pressure sensor(s) 103j can be operably positioned by the wound therapy device 103 such that they can measure the pressure level within the wound dressing 102 and/or the fluid flow path between the negative pressure source 103g and the wound dressing 102. The pressure sensor(s) 102a can be operably positioned by the wound dressing 102 such that they can measure the pressure level within the wound dressing 102. In some embodiments, the pressure sensor(s) 102a may alternatively, or in addition, be disposed within the conduit 106. The processor(s) 103a can be in electrical communication with the pressure sensor(s) 103j and 102a. Further, the processor(s) 103a can be programmed to determine the pressure level within the wound dressing 102 and/or any aspects of the wound therapy device 100 based on the pressure level measured by the pressure sensor(s) 103j and 102a. In some cases, the processor(s) 103a can determine a concentration of nitric oxide within the wound dressing 102 based on the determined pressure level. In some embodiments, the pressure sensor(s) 102a can be optional or not required. An implementation of the pressure sensor(s) 103j can be a pressure sensor 214 described with respect to FIG. 2 herein.

The nitric oxide sensor(s) 103k of the wound therapy device 103 can include one or more nitric oxide sensors configured to measure a concentration of nitric oxide within the wound therapy system 100 and/or any aspects thereof. The nitric oxide sensor(s) 103k can be operably positioned by/within the wound therapy device 103 such that they can measure the concentration of nitric oxide within the wound dressing 102 and/or the fluid flow path between the nitric oxide source 103h and the wound dressing 102. In some embodiments, the nitric oxide sensor(s) may alternatively, or in addition, be operably positioned by/within the conduit 106 and/or by/within the wound dressing 102. The processor(s) 103a can be in electrical communication with the nitric oxide sensor(s) 103k. Further, the processor(s) 103a can be programmed to determine the concentration of nitric oxide within the wound dressing 102 based on the nitric oxide concentration measured by the nitric oxide sensor(s) 103k. An implementation of the nitric oxide sensor(s) 103k can be a nitric oxide sensor 215 described with respect to FIG. 2 herein.

The other sensor(s) 103l of wound therapy device 103 and the other sensor(s) 102b in wound dressing 102 can include one or more of a temperature sensor, an acoustic sensor, a motion sensor, additional nitric oxide sensor(s), and/or any other sensor configured to measure a parameter of the wound therapy system 100 and/or any aspects thereof. The other sensor(s) 103l and 102b can be operably positioned by the wound therapy device 103 and the wound dressing 102, respectively, and can be in electrical communication with the processor(s) 103a. The processor(s) 103a can be programmed to determine a relevant parameter of the wound therapy system 100 based on the parameter measured by such other sensor(s) 103l and 102b. In some embodiments, the conduit 106 can include the other sensor(s) 102b. In some cases, the other sensor(s) 103l and/or 102b can be optional or not required.

FIG. 2 illustrates a schematic flow diagram of the wound therapy system 100 of FIGS. 1A-1D in accordance with some embodiments of this disclosure. As discussed herein and as shown, the wound therapy system 100 can include the wound therapy device 103, the conduit 106, and the wound dressing 102. The wound therapy device 103 can include the negative pressure source 104, the pressure sensor 214, the nitric oxide source 105, the valve 210, and the nitric oxide sensor 215. The wound therapy device 103 can apply negative pressure 204 and deliver nitric oxide 205 to the wound dressing 102 via the conduit 106.

Within the wound therapy device 103 and as shown, the nitric oxide source 105 can connect to the valve 210. As discussed herein, the valve 210 can be a solenoid valve configured to regulate the delivery of nitric oxide 205 to the wound dressing 102. The nitric oxide sensor 215 can be disposed in the fluid pathway between the valve 210 and the wound dressing 102 and measure the concentration of nitric oxide 205 within the wound dressing 102. The controller of the wound therapy device 103 can determine the concentration of nitric oxide within the wound dressing via the nitric oxide sensor 215 and operate the valve 210 to regulate the concentration of nitric oxide.

Further as shown, the negative pressure source 104 of the wound therapy device 103 can connect to the wound dressing 102. The pressure sensor 214 can be disposed within the wound therapy device 103 in the fluid pathway between the wound dressing 102 and the negative pressure source 104 and measure the level of negative pressure 204 within the wound dressing 102. The controller of the wound therapy device 103 can determine the level of negative pressure within the wound dressing via the pressure sensor 214 and operate the negative pressure source 104 (e.g., via control of the pump of the negative pressure source 104 as described herein) to regulate the negative pressure level. In some embodiments, the controller of the wound therapy device 103 can utilize the pressure level of the wound therapy system 100 as measured by the pressure sensor 214 in the regulation of nitric oxide delivery (e.g, by utilizing the pressure level to regulate the source of nitric oxide 105 directly or via operation of the valve 210).

Also shown, the negative pressure source 104 can exhaust fluid (e.g., gas and/or liquid) to atmosphere via exhaust 206 during operation (e.g., during operation of the pump of the negative pressure source 104). In some cases, the exhaust 206 can be utilized as a controlled leak path for allowing fluid within the wound therapy system 100 to exhaust and/or for allowing atmospheric air to enter the wound therapy system 100. Further, in some embodiments the wound therapy device 103 can include an exhaust valve configured to control such exhaust 206. Such an exhaust valve can be a low power active valve, for example, a solenoid valve. In some embodiments, a filter can be included in the exhaust 206 pathway, such as between an outlet of the negative pressure source 104 and atmosphere. The filter can provide filtration of the air prior to venting to the atmosphere. In some embodiments, the filter can be a bacterial filter, odor filter, etc. or any combination thereof. In some embodiments, a dampening component, such as a noise dampening component, can be interposed between the outlet and the atmosphere. The dampening component can reduce the noise generated by the pump of the negative pressure source 104 during operation.

Although the wound therapy system 100, in particular the wound dressing 102, can be configured to fluidically seal the wound site as discussed herein, a leak 202 may occur during use. The leak 202 is shown as introducing atmospheric air into the wound site past the wound dressing 102. The wound therapy system 100 can recognize such a leak 202 (e.g., determined by the controller of the wound therapy device 103) and can provide an alert and/or alarm to the user and/or their healthcare provider so that the leak 202 can be mitigated.

FIG. 3 illustrates operation of the wound therapy system 100 in accordance with some embodiments of this disclosure. Shown is an example plot of the nitric oxide concentration 305 within a wound dressing, such as the wound dressing 102 described herein, in ppm on the positive y axis and the pressure level 304 within the wound dressing in mmHg on the negative y axis through time that can be achieved by the wound therapy system 100.

As shown, before the start of therapy the wound dressing is at a baseline negative pressure level 314 of approximately zero, indicating that the wound dressing is at approximately atmospheric pressure. Also shown, before the start of therapy the wound dressing is at a baseline nitric oxide concentration 315 of approximately zero.

At a first time 351, such as at the beginning of therapy or when resuming therapy after a pause, the controller of the wound therapy system 100 can be programmed to apply negative pressure to the wound dressing to cause the pressure level 304 within the wound dressing to reach a first negative pressure level 324. Such application of negative pressure can be referred to as an initial pump down. The first negative pressure level can be approximately −120 as shown, or any negative pressure level between approximately −80 mmHg and approximately −200 mmHg.

At a second time 352, the controller can be programmed to pause or reduce application of negative pressure to the wound dressing. Further, at the second time 352, the controller can be programmed to deliver nitric oxide to the wound dressing to cause the nitric oxide concentration 305 within the wound dressing to increase from the baseline nitric oxide concentration 315 until a first nitric oxide concentration 325 within the wound dressing is reached at a third time 353. The first nitric oxide concentration 325 can be approximately 90 ppm as shown, or any nitric oxide concentration above approximately 80 ppm. The delivery of nitric oxide to the wound dressing can cause the pressure level 304 within the wound dressing to increase (e.g., become closer to atmospheric pressure) from the first negative pressure level 324 to a second negative pressure level 334. The second negative pressure level can be approximately −60 as shown, or any negative pressure level between approximately −20 mmHg and approximately −120 mmHg. In some cases, the controller can be programmed to determine a pressure level for the first pressure level 324 that will result in a desired second pressure level 334 upon delivery of nitric oxide to the wound dressing based on the increase in pressure the delivery of nitric oxide will cause. Upon reaching the first nitric oxide concentration 325 within the wound dressing at the third time 353, the controller can be programmed to pause or reduce delivery of nitric oxide. As shown in FIG. 3, the paused or reduced delivery of nitric oxide at the third time 353 along with the paused or reduced application of negative pressure at the second time 352 can cause the pressure level 304 and the nitric oxide concentration 305 within the wound dressing to be maintained. The first nitric oxide concentration 325 can correspond to the nitric oxide concentration that promotes antipathogenic activity (also referred to herein as the “high concentration”) as discussed with respect to FIG. 2 above. Further, the duration of time between the third time 353 and the fourth time 354 that the first nitric oxide concentration 325 can be delivered to the wound dressing can correspond to the duration(s) of time described with respect to FIG. 2 above.

At a fourth time 354, the controller can be programmed to apply negative pressure to the wound dressing until a second nitric oxide concentration 335 within the wound dressing is reached at a fifth time 355 and a third negative pressure level 334 within the wound dressing between the first negative pressure level 324 and the second negative pressure level 334 is reached (e.g., the application of negative pressure to the wound dressing can be utilized to reduce the nitric oxide concentration 305 within the wound dressing). The second nitric oxide concentration 344 can be approximately 20 ppm as shown, or any nitric oxide concentration below approximately 80 ppm. The third negative pressure level 344 can be approximately −80 mmHg as shown, or any negative pressure level between approximately −40 mmHg and approximately −200 mmHg. Upon reaching the second nitric oxide concentration 335 within the wound dressing at the fifth time 355, the controller can be programmed to pause or reduce application of negative pressure to the wound dressing. As shown in FIG. 3, the paused or reduced delivery of negative pressure at the fifth time 355 along with the paused or reduced application of nitric oxide at the third time 353 can cause the pressure level 304 and the nitric oxide concentration 305 within the wound dressing to be maintained. The second nitric oxide concentration 335 can correspond to the nitric oxide concentration that promotes tissue repair and vasodilation (also referred to herein as the “low concentration”) as discussed with respect to FIG. 2 above. Further, the duration of time between the fifth time 355 and a sixth time 356 that the second nitric oxide concentration 335 can be delivered to the wound dressing can correspond to the duration(s) of time described with respect to FIG. 2 above.

At the sixth time 356, the controller can be programmed to apply negative pressure to the wound dressing to evacuate nitric oxide from the wound dressing and to cause the pressure level 304 within the wound dressing to decrease (e.g., become farther away from atmospheric pressure) towards the first negative pressure level 324. In such a case, the first negative pressure level can be reached at a seventh time 357. Such application of negative pressure can be referred to as a maintenance pump down. Further, in some cases, such evacuation of nitric oxide from the wound dressing can cause the nitric oxide concentration 305 within the wound dressing to return approximately to the baseline nitric oxide concentration 315, to reach zero ppm, and/or to reach a nitric oxide concentration of less than approximately 30 ppm. From the seventh time 357 forward, the application of negative pressure and the delivery of nitric oxide to the wound dressing by the wound therapy system 100 can be a repeat of or similar to the therapy delivered from the second time 352 to the seventh time 357. In such a case, actions of the controller and the wound therapy system 100 at an eight time 358 can be the same or similar as those at the third time 353, actions of the controller and the wound therapy system 100 at a ninth time 359 can be the same or similar as those at the fourth time 354, actions of the controller and the wound therapy system 100 at a tenth time 360 can be the same or similar as those at the fourth time 354, and so on.

While the exemplary plot of FIG. 3 shows flat lines for the pressure level 304 and the nitric oxide concentration 305 within the wound dressing between various times, this may not be the case in practical application. For example, tissue of the wound can absorb at least some of the delivered nitric oxide, which would cause a drop in the nitric oxide concentration 305 through time. As another example, a leak may occur in the system, which would cause the pressure level 304 within the wound dressing to increase towards atmospheric pressure and cause the nitric oxide concentration 305 to decrease through time. In these and other cases where the pressure level 304 and/or the nitric oxide concentration 305 within the wound dressing drifts or is otherwise influenced by external factors (e.g., changed but not by action of the controller), the controller can be programmed to apply negative pressure and/or deliver nitric oxide as necessary to regulate the pressure level 304 and/or the nitric oxide concentration 305 to the level and/or concentration desired or to within the level and/or concentration range desired. For example, the controller can be programmed to deliver nitric oxide to the wound dressing between the third time 353 and the fifth time 355 to maintain the first nitric oxide concentration 325 within the wound dressing. As another example, the controller can be programmed to deliver nitric oxide to the wound dressing between the fifth time 355 and the sixth time 356 to maintain the second nitric oxide concentration 335 within the wound dressing. In some embodiments, the controller is programmed such that the nitric oxide concentration 305 within the wound dressing takes priority over the pressure level 304 within the wound dressing (e.g., nitric oxide can be the primary therapy with negative pressure being the secondary therapy).

Further, while the exemplary plot of FIG. 3 and its associated description above show and describe a therapy protocol that the wound therapy system 100 can deliver, such example and description is not intended to be limiting. In some cases, it can be desirable for a therapy protocol to change over the course of treatment (e.g., to account for changes in the wound such as by healing). As such, the controller of the wound therapy system 100 can be programmed to deliver other therapy protocols and/or therapy protocols that change through time. For example, the concentration and/or timing of nitric oxide delivery can be varied throughout the wound healing continuum. High concentrations of nitric oxide can be delivered during the early part of the wound healing process to manage microbiology (e.g., antipathogenic activity) while lower concentrations can be delivered subsequently to influence wound healing biology (e.g., activation of tissue repair and vasodilation).

The wound therapy system 100 can determine the concentration of nitric oxide within the wound dressing 102 in any number of ways. In some embodiments, and as discussed herein, the controller of the system can determine the concentration of nitric oxide within the wound dressing 102 via measurements from the one or more nitric oxide sensor(s) 103k (such as nitric oxide sensor 215 shown in FIG. 2). In some cases, the controller of the system can determine the nitric oxide concentration within the wound dressing 102 by monitoring a change in pressure within the wound dressing 102 before and after nitric oxide has been delivered to the wound dressing 102. In such a case, the controller can determine the concentration of nitric oxide within the wound dressing 102 based on known characteristics of the nitric oxide source 105 (such as pressure, volume, and/or concentration), the monitored change in pressure within the wound dressing 102, and the volume of the system. In some implementations, the controller of the system can determine the nitric oxide concentration within the wound dressing 102 by monitoring a timing of nitric oxide delivery to the wound dressing 102. In such implementations, the controller can determine the concentration of nitric oxide within the wound dressing 102 based on known characteristics of the nitric oxide source 105 (such as pressure, volume, and/or concentration), the duration of time nitric oxide was delivered, and the volume of the system. In some embodiments, the controller can determine the concentration of nitric oxide within the wound dressing 102 by one or more of measurements from a nitric oxide sensor, measurements from a pressure sensor, a timing of nitric oxide delivery, and/or determination of the volume of the system. Regardless of how determined, the controller can use the concentration of nitric oxide within the wound dressing 102 to control the delivery of nitric oxide to the wound dressing 102 from the nitric oxide source 105.

The wound therapy system 100 can determine the volume of the system, inclusive of any space between the wound dressing 102 and tissue of the wound (e.g., a volume of the wound enclosed by the wound dressing), in any number of ways. In some embodiments, a volume of the system can be pre-programmed and selected based on a size of wound dressing used. In some cases, the controller can be programmed to determine a volume of the system. Further, the controller can be programmed to determine a volume of the system at any point during therapy, such as prior to or during the delivery of negative pressure to the wound dressing 102 and/or prior to the delivery of nitric oxide to the wound dressing 102. In some embodiments, the volume of the system can be determined before the delivery of therapy to the wound dressing, periodically throughout treatment, and/or upon any resume in therapy after a pause. To determine the volume of the system, the controller can monitor a rate of pressure change or the actual pressure change in the system over time. For example, given a known volume that the pump of the negative pressure source 104 can evacuate from the system, the rate of pressure change or the actual pressure change in the system over time when the pump is in use can be monitored and used to determine the volume of the system.

### Other Variations

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. For instance, the various components illustrated in the figures may be implemented as software or firmware on a processor, controller, ASIC, FPGA, or dedicated hardware. Hardware components, such as processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional or alternative components. These components can include menus, lists, buttons, text boxes, labels, radio buttons, scroll bars, sliders, checkboxes, combo boxes, status bars, dialog boxes, windows, and the like. User interface screens can include additional or alternative information. Components can be arranged, grouped, displayed in any suitable order.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A negative pressure wound therapy system configured for delivery of nitric oxide, comprising:
a wound dressing configured for placement over a wound;
a source of negative pressure configured to be connected to the wound dressing;
a source of nitric oxide configured to be connected to the wound dressing; and
a controller configured to regulate nitric oxide delivery and negative pressure application to the wound dressing, the controller programmed to:
apply negative pressure to the wound dressing to cause a pressure level within the wound dressing to reach a first negative pressure level,
pause or reduce application of negative pressure to the wound dressing and deliver nitric oxide to the wound dressing to increase the pressure level within the wound dressing from the first negative pressure level to a second negative pressure level,
pause or reduce delivery of nitric oxide and apply negative pressure to the wound dressing until a third negative pressure level within the wound dressing between the first negative pressure level and the second negative pressure level is reached,
pause or reduce application of negative pressure to the wound dressing, and
apply negative pressure to the wound dressing to evacuate nitric oxide from the wound dressing and to cause the pressure level within the wound dressing to decrease toward the first negative pressure level.

2. The negative pressure wound therapy system of claim 1, wherein, when the third negative pressure level within the wound dressing is reached, application of negative pressure to the wound dressing comprises maintaining said third negative pressure level within the wound dressing until negative pressure is applied to the wound dressing to evacuate nitric oxide from the wound dressing.

3. The negative pressure wound therapy system of claim 1, wherein, when the third negative pressure level within the wound dressing is reached, the controller is further programmed to deliver nitric oxide to the wound dressing to maintain a concentration of nitric oxide within the wound dressing.

4. The negative pressure wound therapy system of claim 1, wherein the source of nitric oxide comprises a source of pressurized nitric oxide gas.

5. (canceled)

6. The negative pressure wound therapy system of claim 1, wherein the source of nitric oxide comprises a nitric oxide generator.

7. (canceled)

8. (canceled)

9. The negative pressure wound therapy system of claim 1, further comprising a valve configured to control the delivery of nitric oxide to the dressing, wherein the controller is in electrical communication with said valve, and wherein the controller is configured to operate the valve to regulate nitric oxide delivery to the wound dressing.

10. (canceled)

11. (canceled)

12. The negative pressure wound therapy system of claim 1, further comprising an exhaust valve configured to control a supply of atmospheric pressure to the wound dressing, wherein the controller is in electrical communication with said exhaust valve, and wherein the controller is configured to operate the exhaust valve to regulate the supply of atmospheric pressure to the wound dressing.

13. (canceled)

14. (canceled)

15. (canceled)

16. The negative pressure wound therapy system of claim 1, further comprising a housing configured to house the source of negative pressure, the source of nitric oxide, and the controller.

17. (canceled)

18. (canceled)

19. The negative pressure wound therapy system of claim 1, wherein said wound dressing comprises the source of negative pressure, the source of nitric oxide, and the controller.

20. The negative pressure wound therapy system of claim 1, further comprising a pressure sensor configured to measure the pressure level within the wound dressing, wherein the controller is in electrical communication with said pressure sensor, and wherein the controller is further programmed to determine the pressure level within the wound dressing based on the pressure level measured by said pressure sensor.

21. (canceled)

22. The negative pressure wound therapy system of claim 20, wherein the controller is further programmed to determine a concentration of nitric oxide within the wound dressing based on said determined pressure level.

23. The negative pressure wound therapy system of claim 1, wherein the controller is further programmed to determine an amount of time nitric oxide is delivered to the wound dressing and determine a concentration of nitric oxide within the dressing based on said amount of time.

24. The negative pressure wound therapy system of claim 1, further comprising a nitric oxide sensor configured to measure a concentration of nitric oxide within the wound dressing, wherein the controller is in electrical communication with said nitric oxide sensor, and wherein the controller is further programmed to determine the concentration of nitric oxide within the wound dressing based on the concentration of nitric oxide measured by said nitric oxide sensor.

25. (canceled)

26. (canceled)

27. (canceled)

28. The negative pressure wound therapy system of claim 24, further comprising an electronic display in electrical communication with the controller, and wherein the controller is further programmed to provide on said electronic display a graphical representation of the determined concentration of nitric oxide within the wound dressing.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The negative pressure wound therapy system of claim 1, wherein a concentration of nitric oxide within the wound dressing at the first pressure level comprises a range including or between 0 ppm and 30 ppm.

41. (canceled)

42. (canceled)

43. The negative pressure wound therapy system of claim 1, wherein a concentration of nitric oxide within the wound dressing at the second pressure level is 80 ppm or greater.

44. The negative pressure wound therapy system of claim 1, wherein a concentration of nitric oxide within the wound dressing at the third pressure level comprises a range including or between 5 ppm and 80 ppm.

45. The negative pressure wound therapy system of claim 1, wherein a concentration of nitric oxide within the wound dressing at the third pressure level is between a factor of 5 and a factor of 15 less than a concentration of nitric oxide within the wound dressing at the second pressure level.

46. (canceled)

47. The negative pressure wound therapy system of claim 1, wherein the second negative pressure level is maintained for one hour or less.

48.-69. (canceled)

70. A method of operating a negative pressure wound therapy system configured for delivery of nitric oxide, the method comprising:
applying negative pressure from a source of negative pressure to a wound dressing to cause a pressure level within the wound dressing to reach a first negative pressure level;
pausing or reducing application of negative pressure to the wound dressing and delivering nitric oxide from a nitric oxide source to the wound dressing to increase the pressure level within the wound dressing from the first negative pressure level to a second negative pressure level;
pausing or reducing delivery of nitric oxide and applying negative pressure to the wound dressing until a third negative pressure level within the wound dressing between the first negative pressure level and the second negative pressure level is reached;
pausing or reducing application of negative pressure to the wound dressing; and
applying negative pressure to the wound dressing to evacuate nitric oxide from the wound dressing and to cause the pressure level within the wound dressing to decrease toward the first negative pressure level;
wherein the method is performed under control of a controller.
